(19) United States
(12) Patent Application Publication
(10) Pub. No.: US 2026/0091155 A1
(43) Pub. Date: Apr. 2, 2026

(54) ADHESIVE SKIN DRESSING KIT

(71) Applicant: Juan Medrano, Conroe, TX (US)

(72) Inventor: Juan Medrano, Conroe, TX (US)

(21) Appl. No.: 18/904,939

(22) Filed: Oct. 2, 2024

(51) Int. Cl. A61L 15/58

(52) U.S. Cl. CPC A61L 15/58

(57) ABSTRACT

An adhesive skin dressing kit for securing a medical device to a newborn infant includes a pair of storage sheets each comprised of an adhesive resistant material. A plurality of first adhesive pads is each removably adhered to the adhesive resistant material of a respective one of the pair of storage sheets. A plurality of second adhesive pads is each of the plurality of second adhesive pads is removably adhered to the adhesive resistant material of a respective one of the pair of storage sheets. Each of the plurality of second adhesive pads and first adhesive pads has dimensions sufficiently small to enable the each of the plurality of second adhesive pads to be positionable over a respective one of the plurality of first adhesive pads on the newborn infant's face for securing a medical device between the first adhesive pad and the second adhesive pad.

### CROSS-REFERENCE TO RELATED APPLICATIONS

Not Applicable

### STATEMENT REGARDING FEDERALLY SPONSORED RESEARCH OR DEVELOPMENT

Not Applicable

### THE NAMES OF THE PARTIES TO A JOINT RESEARCH AGREEMENT

Not Applicable

### INCORPORATION-BY-REFERENCE OF MATERIAL SUBMITTED ON A COMPACT DISC OR AS A TEXT FILE VIA THE OFFICE ELECTRONIC FILING SYSTEM

Not Applicable

### STATEMENT REGARDING PRIOR DISCLOSURES BY THE INVENTOR OR JOINT INVENTOR

Not Applicable

### BACKGROUND OF THE INVENTION

### (1) Field of the Invention

The disclosure relates to skin dressing kits and more particularly pertains to a new skin dressing kit for securing a medical device to a newborn infant. The device includes a pair of storage sheets and a plurality of first adhesive pads which are removably adhered to a respective storage sheet and a plurality of second adhesive pads which are removably adhered to a respective storage sheet. Each of the first adhesive pads can be adhered to a newborn infant's skin and each of the second adhesive pads can be adhered to a respective first adhesive pad to secure a medical device between the respective first adhesive pad and a respective second adhesive pad.

### (2) Description of Related Art Including Information Disclosed Under 37 CFR 1.97 and 1.98

The prior art relates to skin dressing kits including a variety of medical kit packaging devices that each has a novel construction for storing medical devices in an organized manner and a post-surgical kit which includes a drain suction bulb and wound dressings and medications all contained in a wearable pouch and an adhesive bandage device that includes a bandage and a nonprescription skin medication applied to the bandage. In no instance does the prior art disclose an adhesive skin dressing kit that includes a pair of storage sheets and a plurality of first adhesive pads, each structured to fit on a newborn infant's face, and a plurality of second adhesive pads, each structured to fit on a newborn infant's face for securing a medical device to a newborn infant's skin or for dressing a wound in the newborn infant's skin.

### BRIEF SUMMARY OF THE INVENTION

An embodiment of the disclosure meets the needs presented above by generally comprising a pair of storage sheets each comprised of an adhesive resistant material. A plurality of first adhesive pads is each removably adhered to the adhesive resistant material of a respective one of the pair of storage sheets. A plurality of second adhesive pads is each of the plurality of second adhesive pads is removably adhered to the adhesive resistant material of a respective one of the pair of storage sheets. Each of the plurality of second adhesive pads and first adhesive pads has dimensions sufficiently small to enable the each of the plurality of second adhesive pads to be positionable over a respective one of the plurality of first adhesive pads on the newborn infant's face for securing a medical device between the first adhesive pad and the second adhesive pad.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### BRIEF DESCRIPTION OF SEVERAL VIEWS OF THE DRAWING(S)

The disclosure will be better understood and objects other than those set forth above will become apparent when consideration is given to the following detailed description thereof. Such description makes reference to the annexed drawings wherein:

FIG. 1 is a perspective view of an adhesive skin dressing kit according to an embodiment of the disclosure.

FIG. 2 is a top view of an embodiment of the disclosure.

FIG. 3 is a perspective in-use view of an embodiment of the disclosure.

FIG. 4 is an exploded in-use view of a temperature probe pad and a temperature probe cover of an embodiment of the disclosure.

FIG. 5 is an exploded in-use view of a cheek pad and a cheek cover of an embodiment of the disclosure.

FIG. 6 is an exploded in-use view of a chin pad and a circular cover of an embodiment of the disclosure.

FIG. 7 is an exploded in-use view of a circular pad and a circular cover of an embodiment of the disclosure.

### DETAILED DESCRIPTION OF THE INVENTION

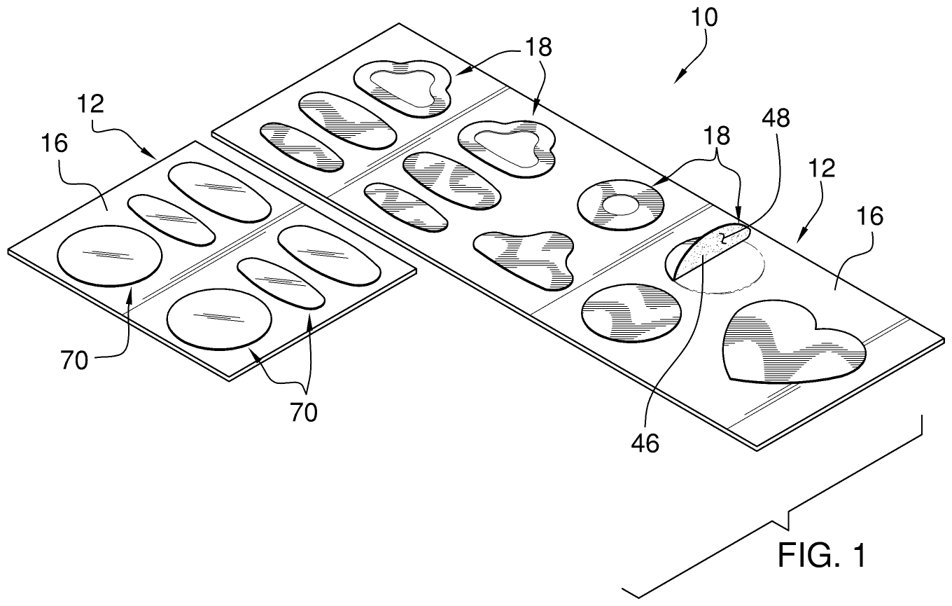

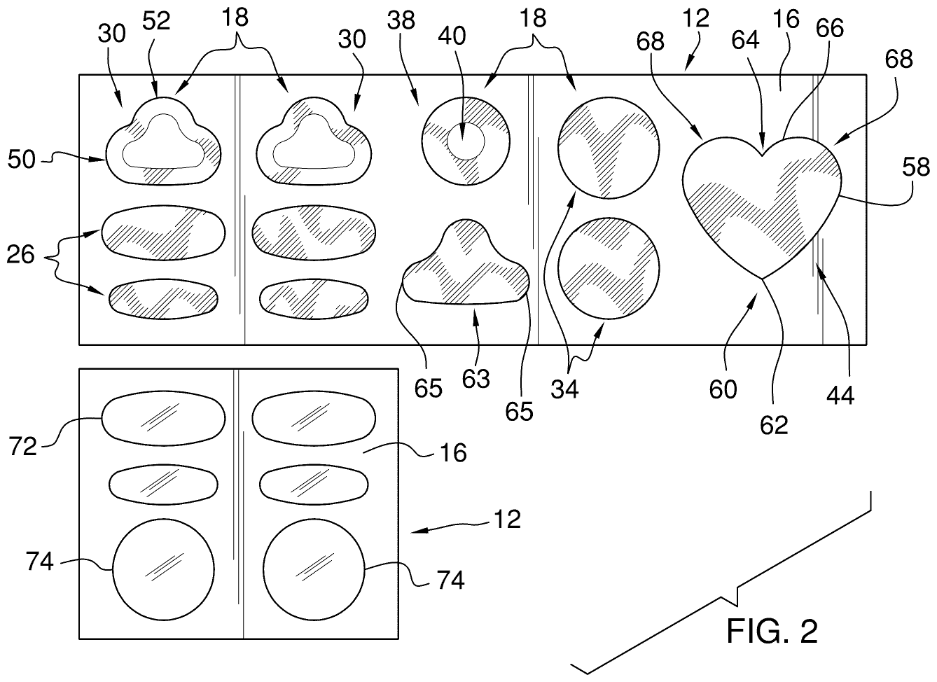

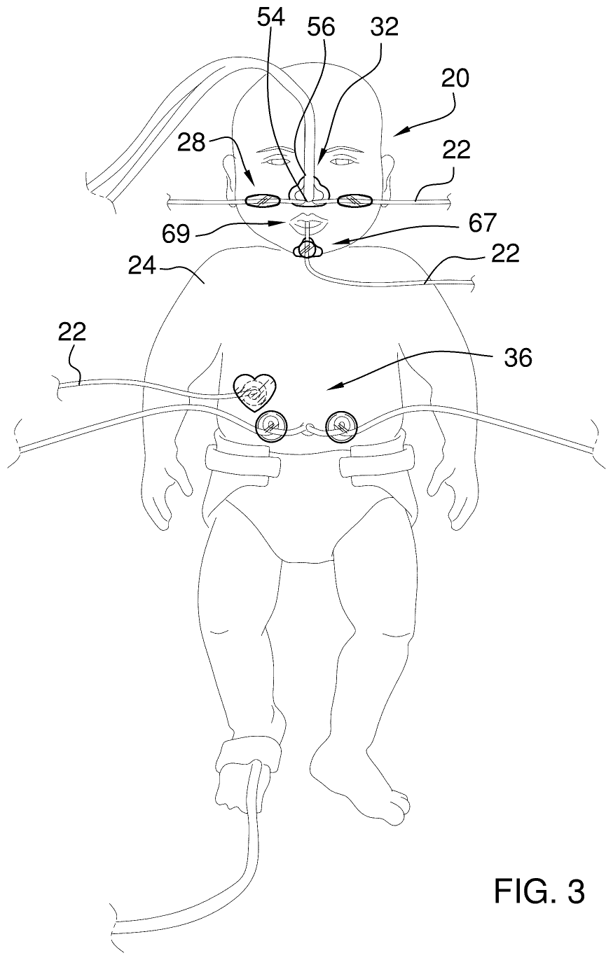

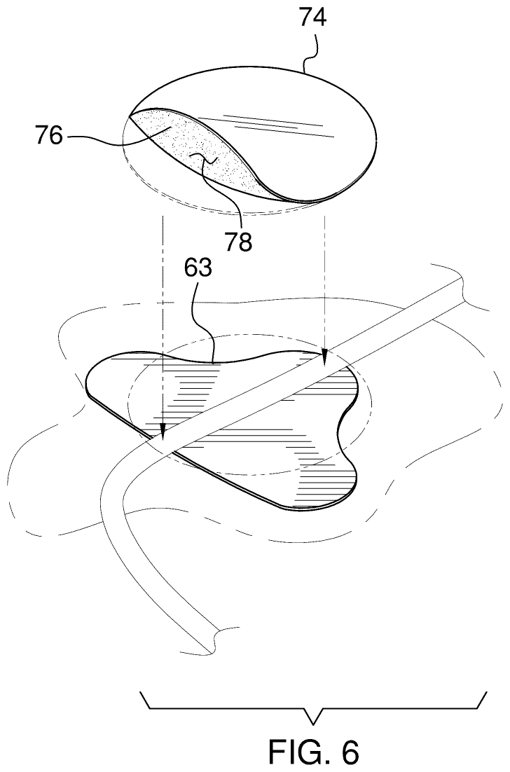

With reference now to the drawings, and in particular to FIGS. 1 through 7 thereof, a new skin dressing kit embodying the principles and concepts of an embodiment of the disclosure and generally designated by the reference numeral 10 will be described.

As best illustrated in FIGS. 1 through 7, the adhesive skin dressing kit 10 generally comprises a pair of storage sheets 12 which is each comprised of an adhesive resistant material. In this way each of the pair of storage sheets 12 can resist adhering to an adhesive. Each of the pair of storage sheets 12 has a top layer 16 which is treated with a coating that is comprised of an adhesive resistant material thereby enabling the top layer 16 of each of the pair of storage sheets 12 to resist adhering to an adhesive. A plurality of first adhesive pads 18 is provided and each of the plurality of first adhesive pads 18 is removably adhered to the adhesive resistant material of a respective one of the pair of storage sheets 12. In this way each of the plurality of first adhesive pads 18 can be removably stored on the respective storage sheet 12.

Each of the plurality of first adhesive pads 18 has dimensions which are sufficiently small to enable the plurality of first adhesive pads 18 to be positionable on a newborn infant's face 20 to secure a medical device 22 to the newborn infant's face 20. The medical device 22 may include, but not be limited to, an oxygen tube or a gastric tube or an electronic thermometer or an intravenous line. Each of the plurality of first adhesive pads 18 is comprised of a resilient material, including but not being limited to, a synthetic fiber or an organic fiber, to inhibit a medical device 22 from injuring the newborn infant's skin 24. Furthermore, each of the plurality of first adhesive pads 18 is comprised of an air permeable material, including but not being limited to, a woven synthetic fiber or a woven organic fiber, to enable the newborn infant's skin 24 the breath beneath the first adhesive pad 18.

The plurality of first adhesive pads 18 includes a set of cheek pads 26 which can each be positioned on the newborn infant's cheek 28. The plurality of first adhesive pads 18 includes a set of nasal pads 30 each structured to form a closed loop thereby enabling the set of nasal pads 30 to be positioned around the newborn infant's nose 32. In this way the nasal pads 30 can inhibit a nasal mask from injuring the newborn infant's skin 24. The plurality of first adhesive pads 18 includes a set of circular pads 34 which can be positioned on the newborn infant's abdomen 36 for inhibiting gastric lines from injuring the newborn infant's skin 24. The plurality of first adhesive pads 18 includes a temperature probe pad 38 which has an opening 40 to enable a temperature probe 42 to contact the newborn infant's skin 24 for reading the temperature of the newborn infant's skin 24. Additionally, the plurality of first adhesive pads 18 includes a temperature probe cover 44 which is positionable over the temperature probe pad 38. In this way the temperature probe cover 44 can secure the temperature probe 42 to the newborn infant's skin 24.

Each of the plurality of first adhesive pads 18 has an adhesive layer 46 which completely covers a bottom surface 48 of the plurality of first adhesive pads 18. Furthermore, the adhesive layer 46 on the bottom surface 48 of each of the plurality of first adhesive pads 18 comprises a non-toxic and releasable adhesive. In this way the adhesive layer 46 on the bottom surface 48 of each of the plurality of first adhesive pads 18 can gently adhere to the newborn infant's skin 24 without injuring the newborn infant's skin 24 when a first adhesive pad 18 which is adhered to the newborn infant's skin 24 is removed from the newborn infant's skin 24.

Each of the set of cheek pads 26 is elongated along a lengthwise axis of the set of cheek pads 26 such that each of the set of cheek pads 26 has an ovoid shape. In this way each of the set of cheek pads 26 extends across a substantial width of the newborn infant's cheek 28 when any of the set of cheek pads 26 is applied to the newborn infant's cheek 28. Each of the closed loops defined by each of the set of nasal pads 30 has a first lobe 50 and a second lobe 52. The first lobe 50 is elongated along a lengthwise axis such that the first lobe 50 has an ovoid shape. In this way the first lobe 50 of the closed loop defined by each of the set of nasal pads 30 can extend around each of the newborn infant's nostrils 54. The second lobe 52 associated with a respective nasal pad 30 extends along an axis which is oriented perpendicular to the lengthwise axis associated with the first lobe 50. In this way the second lobe 52 associated with the respective nasal pad 30 can extend upwardly around the bridge of the newborn infant's nose 56.

The temperature probe cover 44 has a perimeter edge 58 which tapers to a point 60 at a pointed end 62 of the temperature probe cover 44 and which tapers inwardly to a recession 64 on a recessed end 66 of the temperature probe 42. The recession 64 is aligned with the point 60 thereby defining a pair of lobes 68 of the perimeter edge 58 which each curves outwardly between the recessed end 66 and the pointed end 62 such that the temperature probe cover 44 has the ornamental shape of a heart. The bottom surface 48 of the temperature probe cover 44 is comprised of a light reflective material, including but not being limited to a foil type material, to retain heat between the newborn infant's skin 24 and the temperature probe 42 for enhancing accuracy of the temperature probe 42. The set of first adhesive pads 18 includes a chin pad 63 which has a plurality of lobes 65 radiating from a center of the chin pad 63. The chin pad 63 can be positioned on the newborn infant's chin 67 having one of the lobes 65 of the chin pad 63 extending toward the newborn infant's mouth 69.

A plurality of second adhesive pads 70 is provided and each of the plurality of second adhesive pads 70 is removably adhered to the adhesive resistant material of a respective one of the pair of storage sheets 12 thereby enabling each of the plurality of second adhesive pads 70 to be removably stored on the respective storage sheet 12. Each of the plurality of second adhesive pads 70 has dimensions which is sufficiently small to enable the each of the plurality of second adhesive pads 70 to be positionable over a respective one of the plurality of first adhesive pads 18 on the newborn infant's face 20. In this way each of the plurality of second adhesive pads 70 enhances securing the medical device 22 to the newborn infant's face 20 in a manner that protects the newborn infant's face 20 from being injured by the medical device 22.

The plurality of second adhesive pads 70 includes a set of cheek covers 72 and a set of circular covers 74. Each of the set of cheek covers 72 is elongated along a lengthwise axis such that each of the set of cheek covers 72 has an ovoid shape which matches the ovoid shape of a respective one of the set of cheek pads 26. In this way each of the set of cheek covers 72 can secure the medical device 22 between a respective one of the cheek covers 72 and the respective cheek pad 26. Thus, a breathing tube, for example, can be secured to the newborn infant's cheek 28 to inhibit the breathing tube from being dislodged and to also protect the newborn infant's skin 24 from friction injuries or compression injuries that can result from the breathing tube being in contact with the newborn infant's skin 24.

Each of the set of circular covers 74 has a diameter which matches a diameter of the set of circular pads 34. In this way each of the set of circular covers 74 can secure the medical device 22 between a respective one of the circular covers 74 and a respective one of the circular pads 34. Thus, an umbilical venous catheter or an umbilical arterial catheter can be secured to the newborn infant between the respective circular cover 74 and respective circular pad 34. Each of the plurality of second adhesive pads 70 has an adhesive layer 76 completely covering a bottom surface 78 of the plurality of second adhesive pads 70. Furthermore, the adhesive layer 76 on the bottom surface 78 of each of the plurality of second adhesive pads 70 comprises a non-toxic and releasable adhesive. In this way the adhesive layer 76 on the bottom surface 78 of each of the plurality of second adhesive pads 70 can gently adhere to the newborn infant's skin 24 without injuring the newborn infant's skin 24 when a second adhesive pad 70 which is adhered to a respective first adhesive pad 18 on the newborn infant's skin 24 is removed from the respective first adhesive pad 18.

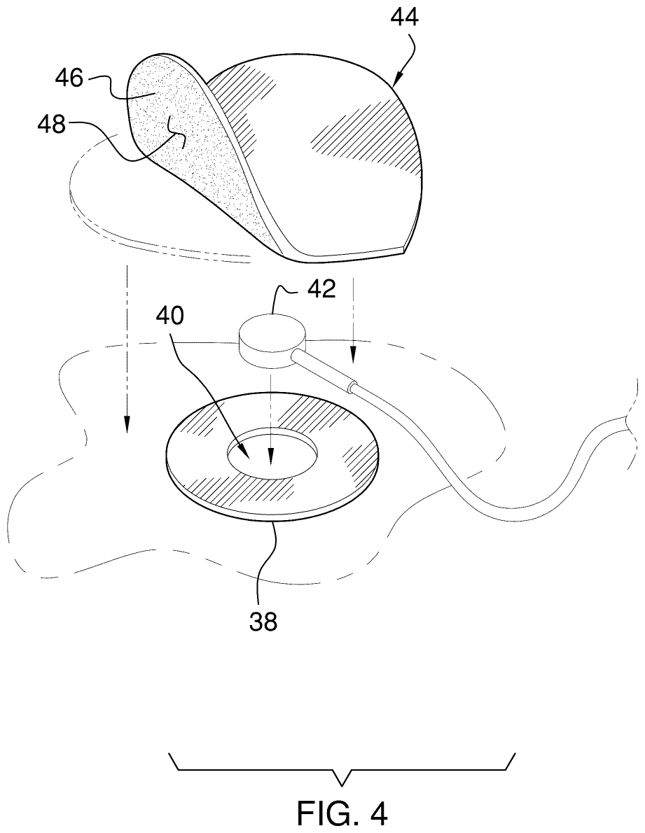

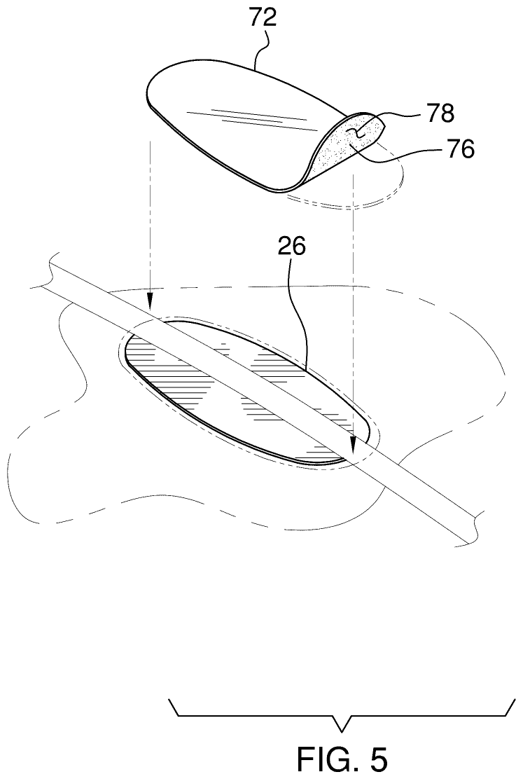

In use, as is shown in FIG. 5, a respective cheek pad 26 is removed from the respective storage sheet 12 and the respective cheek pad 26 is adhered to one of the newborn infant's cheeks 28. A respective cheek cover 72 is removed from the respective storage sheet 12 and the respective cheek cover 72 is adhered to the respective cheek pad 26 such that the medical device 22 is adhered between the respective cheek pad 26 and the respective cheek cover 72. In this way the medical device 22, such as a nasal canula for example, is secured to the newborn infant without the risk of a friction injury or a compression injury to the newborn infant's skin 24. A respective nasal pad 30 is removed from the respective storage sheet 12 and the respective nasal pad 30 is positioned around the newborn infant's nose 32 to inhibit a nasal mask 31 from irritating the newborn infant's skin 24. As is shown in FIG. 4, the temperature probe pad 38 is adhered to the newborn infant's skin 24 and the temperature probe 42 is positioned in the opening 40 in the temperature probe pad 38 to contact the newborn infant's skin 24. The temperature probe cover 44 is removed from the respective storage sheet 12 and the temperature probe cover 44 is adhered to the temperature probe pad 38 to secure the temperature probe 42 in place.

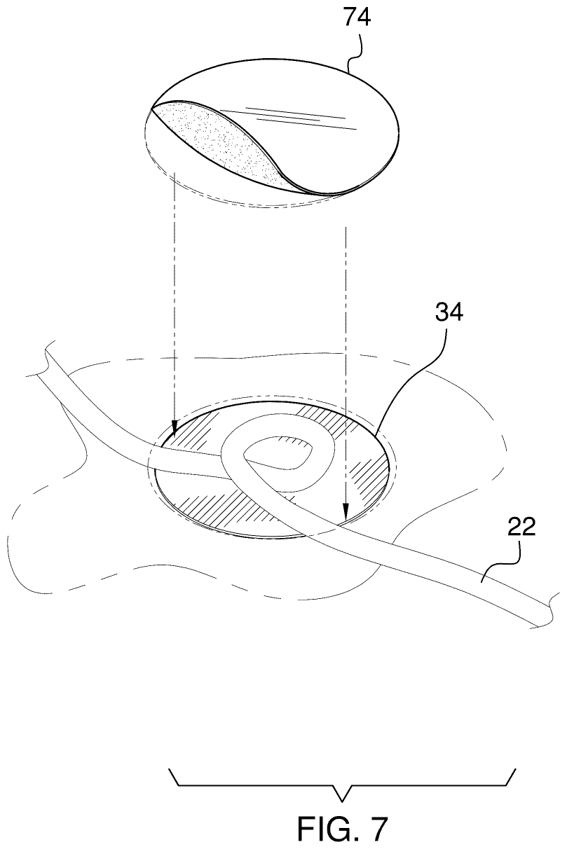

As is shown in FIG. 7, a respective circular pad 34 is removed from the respective storage sheet 12 and the respective circular pad 34 is adhered to the newborn infant's abdomen 36 near the exit point of a gastric tube. A respective circular cover 74 is removed from the respective storage sheet 12 and the respective circular cover 74 is adhered to the respective circular pad 34 to secure the gastric tube between the respective circular pad 34 and the respective circular cover 74. In this way the gastric tube can be secured to the newborn infant without causing a friction injury or a compression injury to the newborn infant's skin 24. Furthermore, the strategically chosen dimensions of the first adhesive pads 18 and the second adhesive pads 70 reduces cut injuries that can result when caregivers must cut traditional sized gauze pads for use on newborn infants.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

I claim:

1. An adhesive skin dressing kit for securing medical devices to the skin of a newborn infant while protecting the newborn infant's skin, said kit comprising:
a pair of storage sheets, each of said pair of storage sheets being comprised of an adhesive resistant material wherein each of said pair of storage sheets is configured to resist adhering to an adhesive;
a plurality of first adhesive pads, each of said plurality of first adhesive pads being removably adhered to said adhesive resistant material of a respective one of said pair of storage sheets thereby enabling each of said plurality of first adhesive pads to be removably stored on said respective storage sheet, each of said plurality of first adhesive pads having dimensions being sufficiently small to enable said plurality of first adhesive pads to be positionable on a newborn infant's face wherein each of said plurality of first adhesive pads is configured to secure a medical device to the newborn infant's face, each of said plurality of first adhesive pads being comprised of a resilient material wherein each of said plurality of first adhesive pads is configured to inhibit a medical device from injuring the newborn infant's skin;
a plurality of second adhesive pads, each of said plurality of second adhesive pads being removably adhered to said adhesive resistant material of a respective one of said pair of storage sheets thereby enabling each of said plurality of second adhesive pads to be removably stored on said respective storage sheet, each of said plurality of second adhesive pads having dimensions being sufficiently small to enable said each of said plurality of second adhesive pads to be positionable over a respective one of said plurality of first adhesive pads on the newborn infant's face wherein each of said plurality of second adhesive pads is configured enhance securing the medical device to the newborn infant's face.

2. The kit according to claim 1, wherein:
said plurality of first adhesive pads includes a set of cheek pads each being configured to be positioned on the newborn infant's cheek;
said plurality of first adhesive pads includes a set of nasal pads each being structured to form a closed loop wherein each of said set of nasal pads is configured to be positioned around the newborn infant's nose for inhibiting a nasal mask from injuring the newborn infant's skin;
said plurality of first adhesive pads includes a set of circular pads wherein each of said set of circular pads is configured to be positioned on the newborn infant's abdomen for inhibiting gastric lines from injuring the newborn infant's skin;
said plurality of first adhesive pads includes a temperature probe pad which has an opening wherein said opening in said temperature probe pad is configured to enable a temperature probe to contact the newborn infant's skin for reading the temperature of the newborn infant's skin; and
said plurality of first adhesive pads includes a temperature probe cover which is positionable over said temperature probe pad wherein said temperature probe cover is configured to secure the temperature probe to the newborn infant's skin.

3. The kit according to claim 2, wherein
each of said set of cheek pads is elongated along a lengthwise axis of said set of cheek pads such that each of said set of cheek pads has an ovoid shape wherein each of said set of cheek pads is configured to extend across a substantial width of the newborn infant's cheek when any of said set of cheek pads is applied to the newborn infant's cheek;
each of said closed loops defined by each of said set of nasal pads has a first lobe and a second lobe;
said first lobe is elongated along a lengthwise axis such that said first lobe has an ovoid shape wherein said first lobe of said closed loop defined by each of said set of nasal pads is configured to extend around each of the newborn infant's nostrils;
said second lobe associated with a respective nasal pad extends along an axis being oriented perpendicular to said lengthwise axis associated with said first lobe wherein said second lobe associated with said respective nasal pad is configured to extend upwardly around the bridge of the newborn infant's nose;
said temperature probe cover has a perimeter edge which tapers to a point at a pointed end of said temperature probe cover and which tapers inwardly to a recession on a recessed end of said temperature probe; and
said recession is aligned with said point thereby defining a pair of lobes of said perimeter edge curving outwardly between said recessed end and said pointed end such that said temperature probe cover has the ornamental shape of a heart.

4. The kit according to claim 2, wherein said bottom surface of said temperature probe cover is comprised of a light reflective material wherein said bottom surface of said temperature probe cover is configured to retain heat between the newborn infant's skin and the temperature probe for enhancing accuracy of the temperature probe.

5. The kit according to claim 1, wherein
each of said pair of storage sheets has a top layer being treated with a coating which is comprised of an adhesive resistant material thereby enabling said top layer of each of said pair of storage sheets to resist adhering to an adhesive;
wherein each of said plurality of first adhesive pads has an adhesive layer completely covering a bottom surface of said plurality of first adhesive pads;
said adhesive layer on said bottom surface of each of said plurality of first adhesive pads comprises a non-toxic and releasable adhesive wherein said adhesive layer on said bottom surface of each of said plurality of first adhesive pads is configured to gently adhere to the newborn infant's skin without injuring the newborn infant's skin when a first adhesive pad which is adhered to the newborn infant's skin is removed from the newborn infant's skin;
said adhesive layer on said bottom surface of each of said plurality of first adhesive pads releasably adheres to said coating on said top surface of said respective storage sheet;
each of said plurality of second adhesive pads has an adhesive layer completely covering a bottom surface of said plurality of second adhesive pads;
said adhesive layer on said bottom surface of each of said plurality of second adhesive pads comprises a non-toxic and releasable adhesive wherein said adhesive layer on said bottom surface of each of said plurality of second adhesive pads is configured to gently adhere to the newborn infant's skin without injuring the newborn infant's skin when a second adhesive pad which is adhered to a respective first adhesive pad on the newborn infant's skin is removed from the respective first adhesive pad; and
said adhesive layer on said bottom surface of each of said plurality of second adhesive pads releasably adheres to said coating on said top surface of said respective storage sheet.

6. The kit according to claim 2, wherein:
said plurality of second adhesive pads includes a set of cheek covers and a set of circular covers;
each of said set of cheek covers is elongated along a lengthwise axis such that each of said set of cheek covers has an ovoid shape which matches said ovoid shape of a respective one of said set of cheek pads wherein each of said set of cheek covers is configured to secure the medical device between a respective one of said cheek covers and said respective cheek pad; and
each of said set of circular covers has a diameter which matches a diameter of said set of circular pads wherein each of said set of circular covers is configured to secure the medical device between a respective one of said circular covers and a respective one of said circular pads.

7. An adhesive skin dressing kit for securing medical devices to the skin of a newborn infant while protecting the newborn infant's skin, said kit comprising:
a pair of storage sheets, each of said pair of storage sheets being comprised of an adhesive resistant material wherein each of said pair of storage sheets is configured to resist adhering to an adhesive, each of said pair of storage sheets having a top layer being treated with a coating which is comprised of an adhesive resistant material thereby enabling said top layer of each of said pair of storage sheets to resist adhering to an adhesive;
a plurality of first adhesive pads, each of said plurality of first adhesive pads being adhered to said adhesive resistant material of a respective one of said pair of storage sheets thereby enabling each of said plurality of first adhesive pads to be removably stored on said respective storage sheet, each of said plurality of first adhesive pads having dimensions being sufficiently small to enable said plurality of first adhesive pads to be positionable on a newborn infant's face wherein each of said plurality of first adhesive pads is configured to secure a medical device to the newborn infant's face, each of said plurality of first adhesive pads being comprised of a resilient material wherein each of said plurality of first adhesive pads is configured to inhibit a medical device from injuring the newborn infant's skin, said plurality of first adhesive pads including a set of cheek pads each being configured to be positioned on the newborn infant's cheek, said plurality of first adhesive pads including a set of nasal pads each being structured to form a closed loop wherein each of said set of nasal pads is configured to be positioned around the newborn infant's nose for inhibiting a nasal mask from injuring the newborn infant's skin, said plurality of first adhesive pads including a set of circular pads wherein each of said set of circular pads is configured to be positioned on the newborn infant's abdomen for inhibiting gastric lines from injuring the newborn infant's skin, said plurality of first adhesive pads including a temperature probe pad which has an opening wherein said opening in said temperature probe pad is configured to enable a temperature probe to contact the newborn infant's skin for reading the temperature of the newborn infant's skin, said plurality of first adhesive pads including a temperature probe cover which is positionable over said temperature probe pad wherein said temperature probe cover is configured to secure the temperature probe to the newborn infant's skin;
wherein each of said plurality of first adhesive pads has an adhesive layer completely covering a bottom surface of said plurality of first adhesive pads, said adhesive layer on said bottom surface of each of said plurality of first adhesive pads comprising a non-toxic and releasable adhesive wherein said adhesive layer on said bottom surface of each of said plurality of first adhesive pads is configured to gently adhere to the newborn infant's skin without injuring the newborn infant's skin when a first adhesive pad which is adhered to the newborn infant's skin is removed from the newborn infant's skin;
wherein each of said set of cheek pads is elongated along a lengthwise axis of said set of cheek pads such that each of said set of cheek pads has an ovoid shape wherein each of said set of cheek pads is configured to extend across a substantial width of the newborn infant's cheek when any of said set of cheek pads is applied to the newborn infant's cheek;
wherein each of said closed loops defined by each of said set of nasal pads has a first lobe and a second lobe, said first lobe being elongated along a lengthwise axis such that said first lobe has an ovoid shape wherein said first lobe of said closed loop defined by each of said set of nasal pads is configured to extend around each of the newborn infant's nostrils, said second lobe associated with a respective nasal pad extending along an axis being oriented perpendicular to said lengthwise axis associated with said first lobe wherein said second lobe associated with said respective nasal pad is configured to extend upwardly around the bridge of the newborn infant's nose;
wherein said temperature probe cover has a perimeter edge which tapers to a point at a pointed end of said temperature probe cover and which tapers inwardly to a recession on a recessed end of said temperature probe, said recession being aligned with said point thereby defining a pair of lobes of said perimeter edge curving outwardly between said recessed end and said pointed end such that said temperature probe cover has the ornamental shape of a heart;
wherein said bottom surface of said temperature probe cover is comprised of a light reflective material wherein said bottom surface of said temperature probe cover is configured to retain heat between the newborn infant's skin and the temperature probe for enhancing accuracy of the temperature probe;
a plurality of second adhesive pads, each of said plurality of second adhesive pads being adhered to said adhesive resistant material of a respective one of said pair of storage sheets thereby enabling each of said plurality of second adhesive pads to be removably stored on said respective storage sheet, each of said plurality of second adhesive pads having dimensions being sufficiently small to enable said each of said plurality of second adhesive pads to be positionable over a respective one of said plurality of first adhesive pads on the newborn infant's face wherein each of said plurality of second adhesive pads is configured enhance securing the medical device to the newborn infant's face, said plurality of second adhesive pads including a set of cheek covers and a set of circular covers, each of said set of cheek covers being elongated along a lengthwise axis such that each of said set of cheek covers has an ovoid shape which matches said ovoid shape of a respective one of said set of cheek pads wherein each of said set of cheek covers is configured to secure the medical device between a respective one of said cheek covers and said respective cheek pad, each of said set of circular covers having a diameter which matches a diameter of said set of circular pads wherein each of said set of circular covers is configured to secure the medical device between a respective one of said circular covers and a respective one of said circular pads; and
wherein each of said plurality of second adhesive pads has an adhesive layer completely covering a bottom surface of said plurality of second adhesive pads, said adhesive layer on said bottom surface of each of said plurality of second adhesive pads comprising a non-toxic and releasable adhesive wherein said adhesive layer on said bottom surface of each of said plurality of second adhesive pads is configured to gently adhere to the newborn infant's skin without injuring the newborn infant's skin when a second adhesive pad which is adhered to a respective first adhesive pad on the newborn infant's skin is removed from the respective first adhesive pad.
